(19) United States
(12) Patent Application Publication
(10) Pub. No.: US 2009/0047379 A1
(43) Pub. Date: Feb. 19, 2009

(54) Benzamide Compounds Useful as High Potency Sweet Taste Enhancers

(76) Inventors: Mark L. Dewis, Matawan, NJ (US); Debra Merrit, Hopewell, NJ (US); Kevin Miller, Middletown, NJ (US); Zhen Chen, Aberdeen, NJ (US); Laurens Reiber, Cambridge (GB)

Correspondence Address: INTERNATIONAL FLAVORS & FRAGRANCES INC., 521 WEST 57TH ST, NEW YORK, NY, 10019, US

(21) Appl. No.: 11/840,334

(22) Filed: Aug. 17, 2007

(51) Int. Cl. A23L 1/236; A23G 4/10; A23L 2/52; C07C 233/65; C07C 229/34; A23L 1/03

(52) U.S. Cl. 426/3; 426/442; 426/538; 426/548; 426/658; 560/41; 562/450; 564/183

(57) ABSTRACT

The present invention relates to novel benzamide compounds may be used to provide desirable property of sweetness and to a foodstuff, chewing gum, medicinal product, toothpaste, alcoholic beverage, aqueous beverage, snack, sauce, confection, baked good, dairy product or cereal.

### FIELD OF INVENTION

This invention relates to novel benzamide compounds useful as high potency flavor molecules for the enhancement of sweet taste quality.

### BACKGROUND OF THE INVENTION

Sweetness can be considered to be the most important taste quality as it is the taste category in which the most compounds have been researched and brought to market. This is probably due to the fact that a craving for foods that are sugar-sweet is among the strongest of human desires, and of the animal kingdom in general. In today's food and beverage industry the most widely used sweetener is sucrose. Sucrose is non-toxic and has a high sweetness quality in terms of a clean, quick onset of sweetness with no lingering or under taste. However, the abundance usage of sucrose and the resulting high caloric intake have caused serious health concerns. In USA more than 20 percent of the adults are obese, and around 17 million people are diabetic with a further 16 million pre-diabetic. Fructose and high fructose corn syrup are also widely used and similar characteristics and concern exist.

In an attempt to eliminate the disadvantages associated with sucrose, a large amount of effort has been devoted to develop artificial sweeteners. The ideal sweet taste compound should possess greater sweetness than sucrose and natural sugars, or enhance sweetness of the natural sugars with preservation of the profile, no color & odor, high solubility in water, high stability at low pH values, high stability with heat, reduced or no calories and no negative physiological effects.

Although some artificial sweeteners, such as Aspartame, Saccharin, Sucralose, Acesulfame K, Oxathiazinone dioxide K, Cyclamic acid, Neotame, and Alitame (along with their salts) have achieved considerable commercial success, none of them could be characterized as “ideal”. Many of them have disadvantages of high cost, lingering sweet taste, bitterness and metallic after taste. The search therefore continues for the ideal sweeteners.

Currently, L-aspartic acid dipeptide derivatives are popular sweeteners. For example, Procter & Gamble has developed a series of α-L-aspartyl-D-phenylglycine and α-L-aspartyl-D-heteroaromatic-substituted glycine esters and amides (represented by the following formula) as sweeteners. Coca-Cola discloses in U.S. Pat. No. 5,286,509 that L-aspartyl-D-α-aminoalkanoyl-(S)-N-α-alkylbenzyl amides of the following formula as artificial sweeteners.

The aspartyl dipeptide derivatives are generally synthesized in a 4-step process which involves coupling of the protected aspartic acid with the protected second amino acid, selective de-protection, necessary modification, and complete removal of protection groups (most likely by catalytic hydrogenation using Pd/C as catalyst). As a result of this multi-step process, the costs of these sweeteners are high. Besides, various dipeptide esters have been known as lacking significant stability at low pH values and this characteristic is detrimental since most carbonated beverages have a pH value between 2.5 and 4.8.

Small molecule sweeteners can be synthesized at low costs. Grant Dubois et al. of Nutrasweet disclose the compounds of the following formula as sugar substitutes in International Patent WO 92/06601.

In a published patent application WO 2005/041684, Senomyx discloses a series of amide compounds of the following formula that could be used as sweet flavoring agents or sweet agent enhancers.

One important discovery of this invention is that unlike in olfaction it is not certain what the relationship is between structure and activity of the sweet molecules. These would also be difficult to model since the sweeteners vary considerably in their structures. Even with Aspartame like dipeptides, which have been studied extensively, it is still difficult to predict that a given compound is sweet and it has been observed repeatedly that a small structural difference can cause dramatic change in the taste. Though some general conclusions can be drawn when one compares the structures of non-sweet molecules with sweet ones, the discovery of new sweeteners is still kind in a hit-or-miss stage. It has therefore been found that since addition, elimination or movement of even a single methyl group within a molecular skeleton can render a molecule sweet, bitter or tasteless, it is impossible to deem any structural modification to a known sweet molecule to obviously lead to another sweet molecule. In fact in most cases it does not.

### SUMMARY OF THE INVENTION

The present invention relates to novel benzamide compounds may be used to provide a desirable property of sweetness to a foodstuff, chewing gum, medicinal product, toothpaste, alcoholic beverage, aqueous beverage, snack, sauce, confection, baked good, dairy product or cereal comprising the step of adding to a foodstuff, chewing gum, medicinal product, toothpaste, alcoholic beverage, aqueous beverage, snack, sauce, confection, baked good, dairy product or cereal at least one benzamide compound defined according to formula 1:

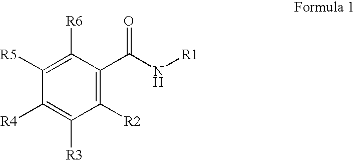

Wherein R₁ is C₁-C₁₀ straight or branched chain alkyl, alkenyl, dienalkyl, phenyl, cycloalkyl, e.g., cyclopropyl, cyclobutyl, cyclopentyl, cyclohexyl, cycloheptyl, etc,; alkyl-substituted cycloalkyls, e.g., 2-methyl-cyclopropyl, 2-methyl-cyclobutyl, 2-methyl-cyclopentyl, 2-methyl-cyclohexyl, 2-methyl-cycloheptyl, 3-methyl-cyclobutyl, 3-methyl-cyclopentyl, 3-methyl-cyclohexyl, 3-methyl-cycloheptyl, 4-methyl-cyclohexyl, 4-methyl-cyclo-heptyl, 2,2-dimethyl-cyclopropyl, 2,3-dimethyl-cyclopropyl, 2,2-dimethyl-cyclobutyl, 2,3-dimethyl-cyclobutyl, 2,4-dimethyl-cyclobutyl, 3,3-dimethyl-cyclobutyl, 2,2,-dimethyl-cyclopentyl, 3,3-dimethyl-cyclopentyl, 2,3-dimethyl-cyclopentyl, 2,4-dimethyl-cyclopentyl, 2,5-dimethyl-cyclopentyl, 3,4-dimethyl-cyclopentyl, 2,2-dimethyl-cyclohexyl, 3,3-dimethyl-cyclohexyl, 4,4-dimethyl-cyclohexyl, 2,3-dimethyl-cyclohexyl, 2,4-dimethyl-cyclohexyl, 2,5-dimethyl-cyclohexyl, 2,6-dimethyl-cyclohexyl, 3,4-dimethyl-cyclohexyl, 3,5-dimethyl-cyclohexyl, etc.; cycloalkenes, e.g., cyclopentanyl, cyclohexenyl, cycloheptenyl, etc.; alkyl-substituted cycloalkenes, e.g., 2-methyl-3-cyclopentenyl, 2-methyl-3-cyclohexenyl, 2-methyl-4-cycloheptenyl, 4-methyl-3-cyclohexenyl, 4-methyl-3-cyclohepenyl, 2,5-dimethyl-3-cyclopentenyl, 3,3-dimethyl-5-cyclohexenyl, etc.; part of the natural amino acids, e.g., alanine, phenylalanine etc. and their esters. R2, R3, R4, R5, and R6 are H, straight or branched chain alkyl containing 1-4 carbon atoms, or alkoxy containing 1-4 carbon atoms.

The present invention relates to novel benzamide compounds may be used to provide a desirable property of sweetness to a foodstuff, chewing gum, medicinal product, toothpaste, alcoholic beverage, aqueous beverage, snack, sauce, confection, baked good, dairy product or cereal comprising the step of adding to a foodstuff, chewing gum, medicinal product, toothpaste, alcoholic beverage, aqueous beverage, snack, sauce, confection, baked good, dairy product or cereal at least one benzamide compound defined according to formula:

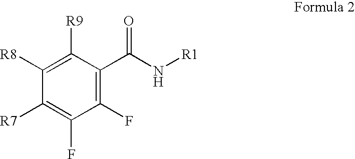

Wherein R₁ is C₁-C₁₀ straight or branched chain alkyl, alkenyl, dienalkyl, phenyl, cycloalkyl, e.g., cyclopropyl, cyclobutyl, cyclopentyl, cyclohexyl, cycloheptyl, etc,; alkyl-substituted cycloalkyls, e.g., 2-methyl-cyclopropyl, 2-methyl-cyclobutyl, 2-methyl-cyclopentyl, 2-methyl-cyclohexyl, 2-methyl-cycloheptyl, 3-methyl-cyclobutyl, 3-methyl-cyclopentyl, 3-methyl-cyclohexyl, 3-methyl-cycloheptyl, 4-methyl-cyclohexyl, 4-methyl-cyclo-heptyl, 2,2-dimethyl-cyclopropyl, 2,3-dimethyl-cyclopropyl, 2,2-dimethyl-cyclobutyl, 2,3-dimethyl-cyclobutyl, 2,4-dimethyl-cyclobutyl, 3,3-dimethyl-cyclobutyl, 2,2,-dimethyl-cyclopentyl, 3,3-dimethyl-cyclopentyl, 2,3-dimethyl-cyclopentyl, 2,4-dimethyl-cyclopentyl, 2,5-dimethyl-cyclopentyl, 3,4-dimethyl-cyclopentyl, 2,2-dimethyl-cyclohexyl, 3,3-dimethyl-cyclohexyl, 4,4-dimethyl-cyclohexyl, 2,3-dimethyl-cyclohexyl, 2,4-dimethyl-cyclohexyl, 2,5-dimethyl-cyclohexyl, 2,6-dimethyl-cyclohexyl, 3,4-dimethyl-cyclohexyl, 3,5-dimethyl-cyclohexyl, etc.; cycloalkenes, e.g., cyclopentanyl, cyclohexenyl, cycloheptenyl, etc.; alkyl-substituted cycloalkenes, e.g., 2-methyl-3-cyclopentenyl, 2-methyl-3-cyclohexenyl, 2-methyl-4-cycloheptenyl, 4-methyl-3-cyclohexenyl, 4-methyl-3-cyclohepenyl, 2,5-dimethyl-3-cyclopentenyl, 3,3-dimethyl-5-cyclohexenyl, etc. part of the natural amino acids, e.g., alanine, phenylalanine etc. and their esters. R7, R8, and R9 are H, straight or branched alkyl containing 1-4 carbon atoms, F, Cl, Br.

In a further embodiment, a sweetening composition is provided comprising at least one of the benzamide compounds of Formula I or Formula II.

In yet another embodiment, ingestible products are provided comprising at least one of the benzamide compounds of Formula I or Formula II.

In another embodiment an ingestible product or oral composition containing an effective amount of benzamide,2,3,4,5,6-pentafluoro-N-(2-methylcyclohexyl)-according in effective amount.

In a further embodiment, a sweetening composition containing a benzamide,2,3,4,5,6-pentafluoro-N-(2-methylcyclohexyl)-1 together with a carrier therefore.

Also in another embodiment, a method is provided for sweetening a substance comprising incorporating an effective amount of benzamide,2,3,4,5,6-pentafluoro-N-(2-methylcyclohexyl)-.

In still a further embodiment sweetening compositions are provided comprising Benzamide,2,3,4,5,6-pentafluoro-N-(2-methylcyclohexyl)—and Benzamide, 2,3,5,6-tetrafluro-4-methyl-N-(2-methylcyclohexyl)-.

In yet a further embodiment a sweetening composition are provided Benzamide,2,3,4,5,6-pentafluoro-N-(2-methylcyclohexyl)-, Benzamide, 2,3,5,6-tetrafluro-4-methyl-N-(2-methylcyclohexyl)—and Sucralose at 60 ppm.

In a further embodiment a sweetening composition is provided comprising Benzamide,2,3,4,5,6-pentafluoro-N-(2-methylcyclohexyl)-, Benzamide, 2,3,5,6-tetrafluro-4-methyl-N-(2-methylcyclohexyl)—and Steavia.

### DETAILED DESCRIPTION OF THE INVENTION

In a preferred embodiment of the invention, the benzamides described by formula 2 have the structure set forth below:

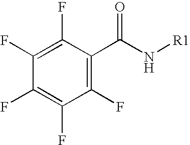

Wherein R₁ is C₁-C₁₀ straight or branched chain alkyl, cycloalkyl, e.g., cyclopropyl, cyclobutyl, cyclopentyl, cyclohexyl, cycloheptyl, etc,; alkyl-substituted cycloalkyls, e.g., 2-methyl-cyclopropyl, 2-methyl-cyclobutyl, 2-methyl-cyclopentyl, 2-methyl-cyclohexyl, 2-methyl-cycloheptyl, 3-methyl-cyclobutyl, 3-methyl-cyclopentyl, 3-methyl-cyclohexyl, 3-methyl-cycloheptyl, 4-methyl-cyclohexyl, 4-methyl-cycloheptyl, etc.

Benzamide compounds of formula 1 were synthesized according to the following scheme:

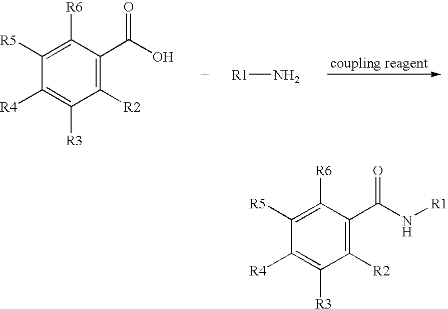

One of the most common methods to prepare an amide is the coupling reaction between a carboxylic acid and an amine in the presence of coupling reagents. Carbodiimides such as N,N′-diisopropylcarbodiimide (DIC), N,N′-dicyclohexylcarbodiimide (DCC), and 1-ethyl-3-(3′-dimethylaminopropyl)carbodiimide hydrochloride (EDAC) are the most popular ones. Besides, phosphonium salts including benzotriazol-1-yl-N-oxy-tris(dimethylamino)phosphonium hexafluorophosphate (BOP) and bensotriazol-1-yl-N-oxy-tris(pyrrolidino)phosphonium hexafluorophosphate (PyBOP) and aminium salts including N-[(dimethylamino)-1H-1,2,3-triazolo[4,5-b]pyridine-1-ylmethylene]-N-methylmethanaminium hexafluorophosphate N-oxide (HATU) and N-[(1 H-benzotriazoll-1-yl)(dimethylamino)methylene]-N-methylmethanaminium hexafluorophosphate N-oxide (HBTU) are widely used too. Other examples of novel benzamide compounds of the present invention defined by formula 1 are listed below:

| Compound # | Structure | Name |
|---|---|---|
| 1 | 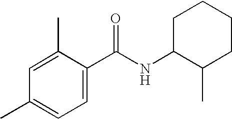 | 2,4-dimethyl-N-(2-methylcyclohexyl)-benzamide |
| 2 | 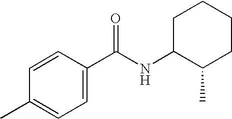 | 4-methyl-N-[2-(S)-methylcyclohexyl)]-benzamide |
| 3 | 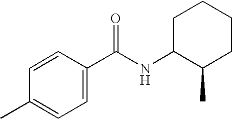 | 4-methyl-N-[2-(R)-methylcyclohexyl)]-benzamide |
| 4 | 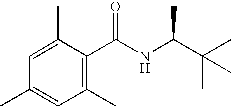 | 2,4,6-trimethyl-N-[(1S)-1,2,2-trimethylpropyl]-benzamide |
| 5 | 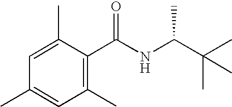 | 2,4,6-trimethyl-N-[(1R)-1,2,2-trimethylpropyl]-benzamide |
| 6 | 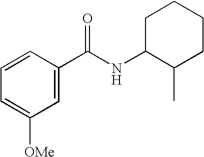 | 3-methoxy-N-(2-methylcyclohexyl)-benzamide |
| 7 | 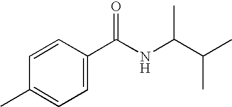 | N-(1,2-dimethylpropyl)-4-methyl-benzamide |
| 8 | 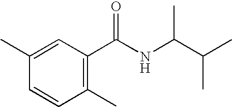 | N-(1,2-dimethylpropyl)-2,5-methyl-benzamide |
| 9 | 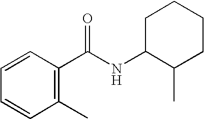 | 2-methyl-N-(2-methylcyclohexyl)-Benzamide |
| 10 | 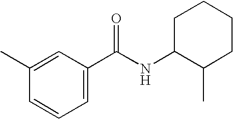 | 3-methyl-N-(2-methylcyclohexyl)-benzamide |
| 11 | 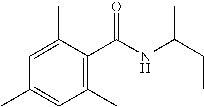 | N-(1-methylpropyl)-2,4,6-trimethyl-benzamide |
| 12 | 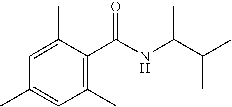 | N-(1,2-dimethylpropyl)-2,4,6-trimethyl-benzamide |
| 13 | 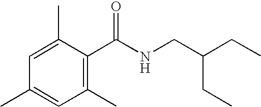 | N-(2-ethylbutyl)-2,4,6-trimethyl-benzamide |
| 14 | 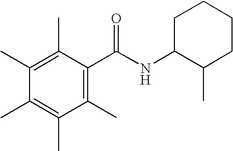 | N-(2-methylcyclohexyl)-2,3,4,5,6-pentamethyl-benzamide |
| 15 | 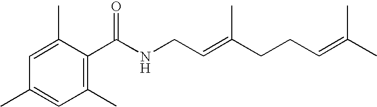 | N-[(2E)-3,7-dimethyl-2,6-octadienyl]-2,4,6-trimethyl-benzamide |
| 16 | 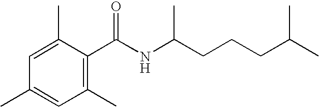 | N-(1,5-dimethylhexyl)-2,4,6-trimethyl-benzamide |
| 17 | 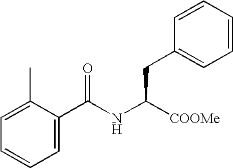 | N-(2-methylbenzoyl)-L-phenylalanine methyl ester |
| 18 | 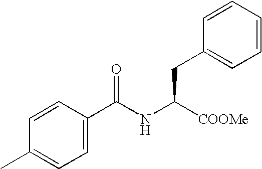 | N-(4-methylbenzoyl)-L-phenylalanine methyl ester |
| 19 | 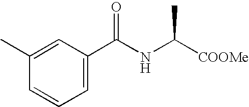 | N-(3-methylbenzoyl)-L-alanine methyl ester |
| 20 | 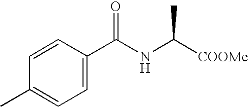 | N-(4-methylbenzoyl)-L-alanine methyl ester |
| 21 | 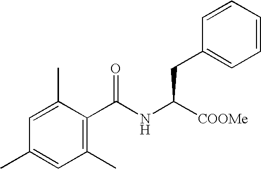 | N-(2,4,6-trimethylbenzoyl)-L-phenylalanine methylester |
| 22 | 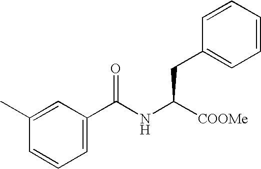 | N-(2-methylbenzoyl)-L-alanine methyl ester |
| 23 | 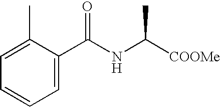 | N-(2-methylbenzoyl)-L-alanine methyl ester |
| 24 | 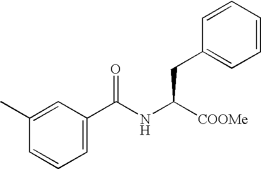 | N-(3-methylbenzoyl)-L-phenylalanine methyl ester |
| 25 | 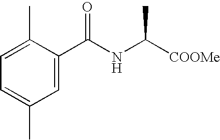 | N-(2,5-dimethylbenzoyl)-L-alanine methyl ester |
| 26 | 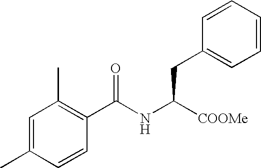 | N-(2,4-dimethylbenzoyl)-L-phenylalanine methyl ester |
| 27 | 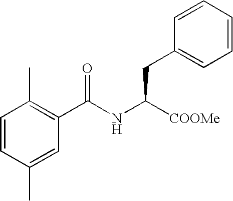 | N-(2,5-dimethylbenzoyl)-L-phenylalanine methyl ester |
| 28 | 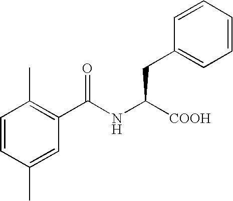 | N-(2,5-dimethylbenzoyl)-L-phenylalanine |
| 29 | 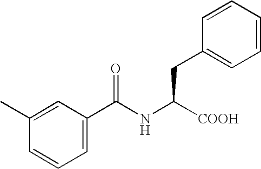 | N-(3-methylbenzoyl)-L-phenylalanine |
| 30 | 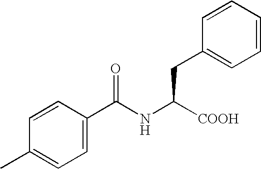 | N-(4-methylbenzoyl)-L-phenylalanine |
| 31 | 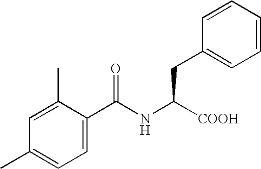 | N-(2,4-dimethylbenzoyl)-L-phenylalanine |
| 32 | 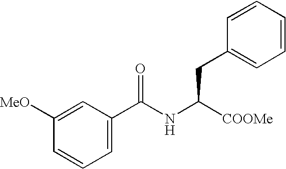 | N-(3-methoxybenzoyl)-L-phenylalanine methyl ester |
| 33 | 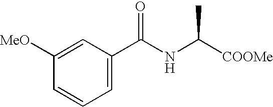 | N-(3-methoxybenzoyl)-L-alanine methyl ester |
| 34 | 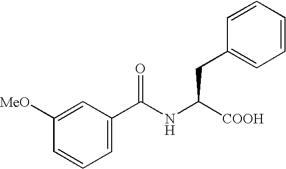 | N-(3-methoxybenzoyl)-L-phenylalanine |
| 35 | 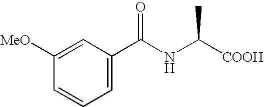 | N-(3-methoxybenzoyl)-L-alanine |

Benzamide compounds of formula 2 were synthesized according to the following scheme:

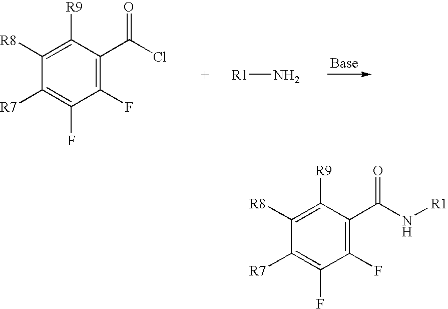

In the presence of an organic base, such as triethyl amine (Et₃N) and N-methyl morpholine (NMM), an acid chloride will react with an amine to form an amide compound. The following examples are provided for the guidance of the reader, and represent a preferred method for preparing the compounds exemplified herein and it is apparent to one skilled in the art that other routes may be employed to prepare these compounds.

Other examples of novel benzamide compounds of the present invention defined by formula 2 are listed below:

| Compound # | Structure | Name |
|---|---|---|
| 36 | 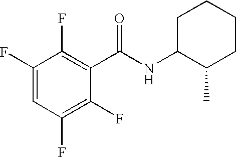 | N-[(2S)-2-methylcyclohexyl)-2,3,5,6-tetrafluoro-benzamide |
| 37 | 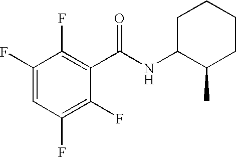 | N-[(2R)-2-methylcyclohexyl)-2,3,5,6-tetrafluoro-benzamide |
| 38 | 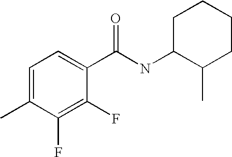 | 2,3-difluoro-4-methyl-N-(2-methylcyclohexyl)-benzamide |
| 39 | 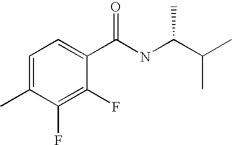 | N-[(1R)-1,2-dimethylpropyl]-2,3-difluoro-4-methyl-benzamide |
| 40 | 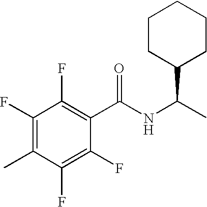 | (R)-N-1-cyclohexylethyl-2,3,5,6-tetrafluoro-4-methyl-benzamide |
| 41 | 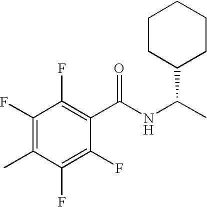 | (S)-N-1-cyclohexylethyl-2,3,5,6-tetrafluoro-4-methyl-benzamide |
| 42 | 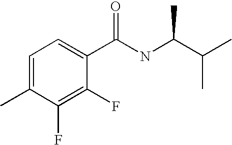 | N-[(1S)-1,2-dimethylpropyl]-2,3-difluoro-4-methyl-benzamide |
| 43 | 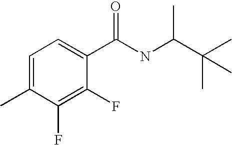 | 2,3-difluoro-4-methyl-N-(1,2,2-trimethylpropyl)-benzamide |
| 44 | 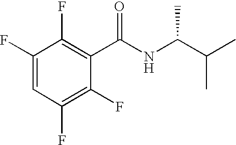 | N-[(1S)-1,2-dimethylpropyl)-2,3,5,6-tetrafluoro-benzamide |
| 45 | 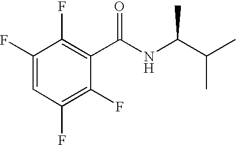 | N-[(1R)-1,2-dimethylpropyl)-2,3,5,6-tetrafluoro-benzamide |
| 46 | 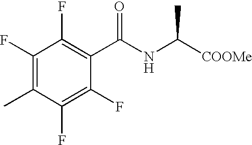 | N-(2,3,5,6-tetrafluoro-4-methylbenzoyl)-L-alaninemethyl ester |
| 47 | 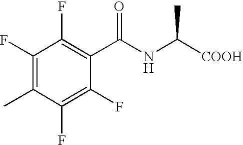 | N-(2,3,5,6-tetrafluoro-4-methylbenzoyl)-L-alanine |
| 48 | 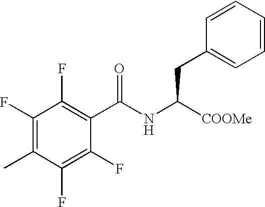 | N-(2,3,5,6-tetrafluoro-4-methylbenzoyl)-L-phenylalanine methyl ester |
| 49 | 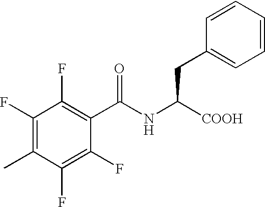 | N-(2,3,5,6-tetrafluoro-4-methylbenzoyl)-L-phenylalanine |
| 50 | 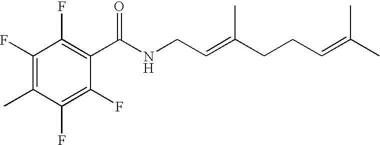 | N-[(2E)-3,7-dimethyl-2,6-octadienyl]-2,3,5,6-tetrafluoro-4-methyl-benzamide |
| 51 | 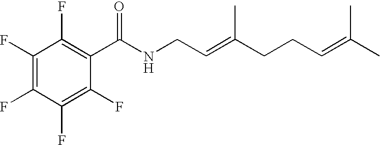 | N-[(2E)-3,7-dimethyl-2,6-octadienyl]-2,3,4,5,6-pentafluoro-benzamide |
| 52 | 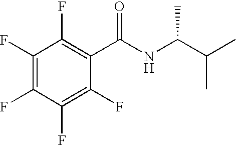 | N-[(1R)-1,2-dimethylpropyl]-2,3,4,5,6-pentafluoro-benzamide |
| 53 | 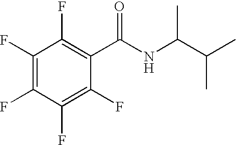 | N-(1,2-dimethylpropyl)-2,3,4,5,6-pentafluoro-benzamide |
| 54 | 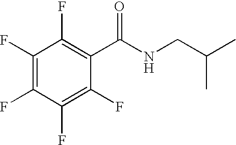 | N-(2-methylpropyl)-2,3,4,5,6-pentafluoro-benzamide |
| 55 | 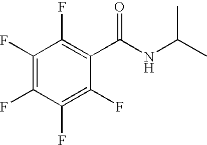 | N-(1-methylethyl)-2,3,4,5,6-pentafluoro-benzamide |
| 56 | 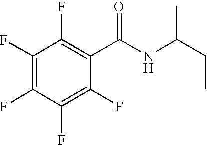 | N-(1-methylpropyl)-2,3,4,5,6-pentafluoro-benzamide |
| 57 | 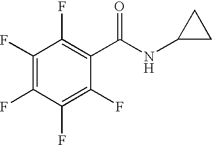 | N-cyclopropyl-2,3,4,5,6-pentafluoro-benzamide |
| 58 | 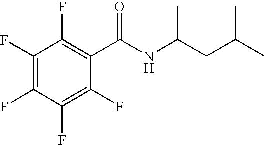 | N-(1,3-dimethylbutyl)-2,3,4,5,6-pentafluoro-benzamide |
| 59 | 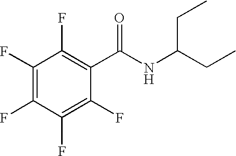 | N-(1-ethylpropyl)-2,3,4,5,6-pentafluoro-benzamide |
| 60 | 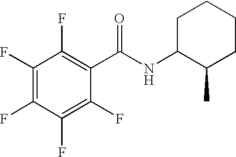 | N-[(2R)-2-methhylcyclohexyl]-2,3,4,5,6-pentafluoro-benzamide |
| 61 | 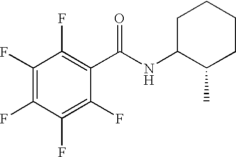 | N-[(2S)-2-methylcyclohexyl]-2,3,4,5,6-pentafluoro-benzamide |
| 62 | 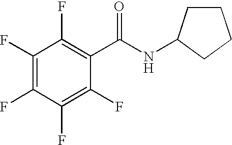 | N-cyclopentyl-2,3,4,5,6-pentafluoro-benzamide |
| 63 | 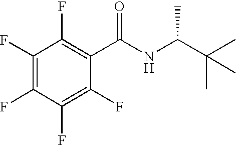 | 2,3,4,5,6-pentafluoro-N-[(1R)-1,2,2-trimethylpropyl]-benzamide |
| 64 | 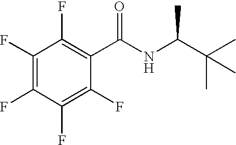 | 2,3,4,5,6-pentafluoro-N-[(1S)-1,2,2-trimethylpropyl]-benzamide |
| 65 | 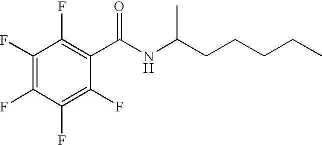 | N-(1-methylhexyl)-2,3,4,5,6-pentafluoro-benzamide |

The following examples are provided for the guidance of the reader, and represent a preferred method for preparing the compounds exemplified herein. It is apparent that one skilled in the art may employ other routes to prepare these compounds.

The benzamide compounds of the present invention may used in combination with artificial sweeteners and natural sugars such as sucrose, glucose, fructose, lactose, maltose, erythritol, isomalt, lactitol, mannitol, sorbitol, xylitol, flavanoid, protein sweetener, aspartame, saccharin, acesulfame-k, cyclamate, sucralose, steavia and alitame, and a mixture thereof and mixtures thereof.

In one embodiment, the compound of the Structure I,

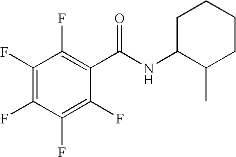

also known by one skilled in the art benzamide,2,3,4,5,6-pentafluoro-N-(2-methylcyclohexyl)-, may be used in combination with artificial sweeteners and natural sweetening compositions such as sucrose, glucose, fructose, lactose, maltose, erythritol, isomalt, lactitol, mannitol, sorbitol, xylitol, flavanoid, protein sweetener, aspartame, saccharin, acesulfame-k, cyclamate, sucralose, steavia, luo han guo and alitame, and a mixture thereof and mixtures thereof.

An ingestible product or oral composition containing an effective amount of benzamide,2,3,4,5,6-pentafluoro-N-(2-methylcyclohexyl)—according in effective amount. A sweetening composition containing a benzamide,2,3,4,5,6-pentafluoro-N-(2-methylcyclohexyl)-1 together with a carrier therefore. A method is also provided for sweetening a substance comprising incorporating an effective amount of benzamide,2,3,4,5,6-pentafluoro-N-(2-methylcyclohexyl)-.

Various sweetener blends are also provided containing Benzamide,2,3,4,5,6-pentafluoro-N-(2-methylcyclohexyl)—at 2 ppm and Benzamide, 2,3,5,6-tetrafluro-4-methyl-N-(2-methylcyclohexyl)—at 2 ppm. The taste comments for the blend was sweetness upfront, drying, some mouthwatering, lingers artificial sweetener.

Various sweetener blends are also provided containing sweetener Blend 2 contains Benzamide,2,3,4,5,6-pentafluoro-N-(2-methylcyclohexyl)—at 2 ppm, Benzamide, 2,3,5,6-tetrafluro-4-methyl-N-(2-methylcyclohexyl)—at 2 ppm and Sucralose at 60 ppm. The taste comments were sweet upfront, natural sugar taste, mouthwatering, lingers sweet, well rounded profile.

Various sweetener blends are also provided containing Sweetener Blend 3 contains Benzamide,2,3,4,5,6-pentafluoro-N-(2-methylcyclohexyl—at 2 ppm, Benzamide, 2,3,5,6-tetrafluro-4-methyl-N-(2-methylcyclohexyl—at 2ppm and Steavia at 1000 ppm.

A method for modulating the taste of a consumable product comprising providing at least one consumable product, or a precursor thereof, and combining the consumable or medicinal product or precursor thereof with at least a sweet flavor-modulating amount of the compound structure I:

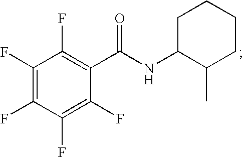

wherein the modified consumable or medicinal product is a food for human and animal consumption.

The modified consumable or medicinal products is selected from the group consisting of confectionaries, bakery products, ice cream, dairy products, sweet and savory snack bars, meal replacement products, ready meals, soups, pastas, noodles, canned foods, frozen foods, dried foods, chilled foods, uncooked food, a fully or partially cooked food, a cooking aid product, meal solution product, a meal enhancement product, a seasoning or seasoning blend, oils and fats, baby foods, spreads, a cake, cookie, pie, candy, chewing gum, gelatin, ice cream, sorbet, pudding, jam, jelly, salad dressing, condiment, cereal, canned fruit, fruit sauces, a beverage, a beverage mix, a beverage concentrate, a soda, juice, an alcoholic beverage or an oral hygiene product.

The compound is present in the modified consumable or medicinal product in a concentration from about 0.001 ppm to about 100 ppm. Preferably, the modified consumable or medicinal product in a concentration from about 0.01 ppm to about 30 ppm, more preferably, the compound may be present in the modified consumable or medicinal product in a concentration from about 0.05 ppm to about 15 ppm, and most preferably the compound is present in the modified consumable or medicinal product in a concentration from about 0.1 ppm to about 5 ppm.

It is a further embodiment of the water solution comprising the sweet flavor modifying amount of the compound and amount 6% of sucrose or 60,000 ppm has a sweetener taste than a control water solution comprising 6% of sucrose or 60,000 ppm, as judged by the majority of experienced taste testers.

Furthermore, in another embodiment a water solution comprising the sweet flavor modifying amount of the compound and amount 6% or 60,000 ppm of a 50:50 mixture of sucrose and fructose has a sweetener taste than a control water solution comprising 6% or 60,000 ppm of a 50:50 mixture of sucrose and fructose, as judged by the majority of experienced taste testers.

A water solution is provided comprising the sweet flavor modifying amount of the compound and from about 0.05% (500 ppm) to about 0.5% (5,000 ppm) of aspartame has a sweetener taste than a control water solution comprising from about 0.05% to about 0.5% of aspartame, as judged by the majority of experienced taste testers.

A water solution is provided comprising the sweet flavor modifying amount of the compound and 0.05% (500 ppm) to about 0.5% (5,000 ppm) of acesulfame-K has a sweetener taste than a control water solution comprising 0.05% to about 0.5% of acesulfame-K, as judged by the majority of experienced taste testers.

A water solution is provided comprising the sweet flavor modifying amount of the compound and 0.05% (500 ppm) to about 0.5% (5,000 ppm) of cyclamate has a sweetener taste than a control water solution comprising 0.05% to about 0.5% of cyclamate, as judged by the majority of experienced taste testers.

A water solution comprising the sweet flavor modifying amount of the compound and 0.002% (20 ppm) to about 0.01% (100 ppm) of of sucralose has a sweetener taste than a control water solution comprising 0.002% to about 0.01% of sucralose, as judged by the majority of experienced taste testers.

A water solution comprising the sweet flavor modifying amount of the compound and 0.01% (100 ppm) to about 0.1% (1000 ppm) of stevia has a sweetener taste than a control water solution comprising 0.0 1% to about 0.1% of stevia, as judged by the majority of experienced taste testers.

A water solution comprising the sweet flavor modifying amount of the compound and 0.005% (50 ppm) to about 0.1% (100 ppm) of stevia has a sweetener taste than a control water solution comprising 0.005%to about 0.1% of stevia, as judged by the majority of experienced taste testers.

A water solution comprising the sweet flavor modifying amount of the compound and 0.005% (50 ppm) to about 0.1% (100 ppm) of luo han guo has a sweetener taste than a control water solution comprising 0.005%to about 0.1% of luo han guo, as judged by the majority of experienced taste testers.

The modified consumable or medicinal products has a sweeter taste as compared to the consumable or medicinal product prepared without the compound, as judged by the majority of a panel of experienced taste testers.

The compound is or can be determined to be generally recognized as safe for use in specific food products at a specified concentration in the finished product.

Another method of reporting the level of the compounds of the invention in the perfumed composition, i.e., the compounds, as parts per million (ppm) of the materials added to impart the desired fragrance. The compounds of the invention can range widely from 0.005 to about 10 ppm of the perfumed composition, and preferably from about 0.1 to about 5 ppm. Those with skill in the art will be able to employ the desired level of the compounds of the invention to provide the desired fragrance and intensity.

All U.S. patents and patent applications cited herein are incorporated by reference as if set forth herein in their entirety.

The following are provided as specific embodiments of the present invention. Other modifications of this invention will be readily apparent to those skilled in the art, without departing from the scope of this invention. Upon review of the foregoing, numerous adaptations, modifications and alterations will occur to the reviewer. These adaptations, modifications, and alterations will all be within the spirit of the invention. Accordingly, reference should be made to the appended claims in order to ascertain the scope of the present invention.

As used herein all percentages are weight percent, ppm stands for parts per million, g stands for grams, ml stands for microliter and mmol stands for micromoles. IFF is meant to be understood as International Flavors & Fragrances Inc., New York, N.Y., USA.

### EXAMPLE 1

### 4-methyl-N-(2-methylcyclohexyl)-benzamide

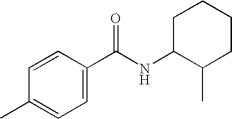

Dissolve 2.04 g (15 mmol) (micromoles) of 4-methyl benzoic acid and 2.70 g(grams) (20 mmol) of N-hydroxybenzoiazole in 20 ml of N,N-dimethylformamide. Add 3.83 g (20 mmol) of N-(3-dimethylaminopropyl)-N′-ethylcarbodiimide hydrochloride to the mixture and stir at room temperature for 15 minutes. 1.70g (15 mmol) of 2-methylcyclohexyl amine was added to the reaction mixture and stir overnight. 100 ml (milliliters) of dichloromethane was added to the mixture and wash it successively with 0.1 N hydrochloric acid, brine, saturated aqueous sodium bicarbonate, and brine. After drying with magnesium sulfate, filtration, concentration under vacuum, and recrystallization (ethyl acetate/hexanes), 1.8 g of white solid was obtained in a yield of 52%. ¹H NMR (CDCl₃) δ 0.94 (d, J=6.96 Hz, 30% of 3H), 0.98 (d, J=6.53 Hz, ˜70% of 3H), 1.11-1.31 (m, 2H), 1.31-1.50 (m, 2H), 1.52-1.73 (m, 1H), 1.74-1.86 (m, 2H), 1.93-2.14 (m, 1H), 2.17-2.25 (m, 1H), 2.37 (2s, 3H), 3.67-3.74 (m, ˜70% of 1H), 4.24-4.29 (m, 30% of 1H), 5.81 (br.s, ˜70% of 1H), 6.09 (br.s, 30% of 1H), 7.22 (d, J=7.85 Hz, 2H), 7.65 (d, J=8.10 Hz, 2H). MS (232.2, M+H).

### EXAMPLE 2

### 2,4,6-trimethyl-N-(1,2,2-trimethylpropyl)-benzamide

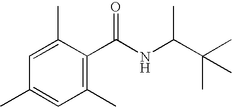

The title compound was prepared in a similar manner to Example 1 using 2, 4, 6-trimethyl benzoic acid and 1, 2, 2-trimethylpropyl amine. A white solid was obtained in a 73.0% yield. ¹H NMR (CDCl₃) δ 0.96 (s, 9H), 1.17 (d, J=6.77 Hz, 3H), 2.27 (s, 3H), 2.30 (s, 6H), 4.07-4.13 (m, 1H), 5.45 (br.s, 1H), 6.84 (s, 2H). MS (248.2, M+H).

### EXAMPLE 3

### N-(2,4-dimethylbenzoyl)-L-alanine methyl ester

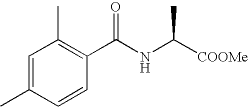

Dissolve 1.50 g (10 mmol) of 2,4-dimethylbenzoic acid and 1.62 g (12 mmol) of N-hydroxybenzoiazole in 20 ml of N, N-dimethylformamide. Start stirring and the mixture was cooled to 0° C. Add 2.30 g (12 mmol) of N-(3-dimethylaminopropyl)-N′-ethylcarbodiimide hydrochloride and 1.32 ml of N-methyl morpholine to the mixture. After stirring 15 minutes remove the cooling bath, add 1.67 g (12 mmol) of L-alanine methyl ester hydrochloride and 1.32 ml of N-methyl morpholine and stir overnight at room temperature. Remove the solvent under vacuum and dissolve the residue in 150 ml of ethyl acetate. Wash the organic layer successively with brine, saturated aqueous sodium bicarbonate, brine, 0. 1 N hydrochloric acid, and brine. After drying with magnesium sulfate, filtration, concentration under vacuum, and recrystallization (ethyl acetate/hexanes), 1.47 g of white solid was obtained in a yield of 62.6%.

¹H NMR (CDCl₃) δ 1.50 (d, J=7.14 Hz, 47% of 3H), 1.51 (d, J=7.16 Hz, 53% of 3H), 2.33 (s, 3H), 2.43 (s, 3H), 3.78 (2s, 3H), 4.77-4.80(m, 1H), 6.29 (br.s, 1H), 7.01 (d, J=7.75 Hz, 1H), 7.04 (s, 1H), 7.32 (d, J=7.66 Hz, 1H). MS (236.1, M+H).

### EXAMPLE 4

### N-(2, 4-dimethylbenzoyl)-L-alanine

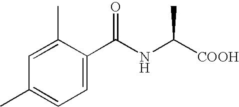

0.70 g (3 mmol) of N-(2,4-dimethylbenzoyl)-L-alanine methyl ester was dissolved in 5 ml of methanol and 5 ml of tetrahydrofuran. Add the solution of 0.50 g (9 mmol) of potassium hydroxide in 2 ml of water to the reaction mixture drop-wise. Stir overnight at room temperature after addition. Remove solvent under vacuum and dissolve the residue in 20 ml of ethyl ether and 40 ml of brine. Separate and acidify the water layer to pH 2 with concentrated hydrochloric acid, white precipitation appeared. Extract the mixture with 50 ml of ethyl acetate and wash the organic layer with brine. After drying with magnesium sulfate, filtration, concentration under vacuum, and recrystallization (ethyl acetate/hexanes), 0.36 g of white solid was obtained in a yield of 54.5%. ¹H NMR (CDCl₃) δ 1.34 (d, J=7.36 Hz, 3H), 2.29 (s, 3H), 2.31 (s, 3H), 4.35 (pentet, J=7.33 Hz, 1H), 7.03 (d, 1H), 7.04 (s, 1H), 7.25 (d, J=7.60 Hz, 1H), 8.39 (d, J=7.26 Hz, 1H). MS (222.1, M+H).

### EXAMPLE 5

### N-[(S)-1,2-dimethylpropyl-2,3,4,5,6-pentafluoro-benzamide

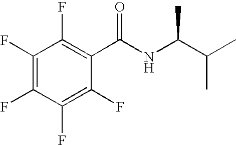

1.31 g (15 mmol) of (S)-3-methyl-2-butylamine and 2.02 g (20 mmol) of triethyl amine were dissolved in 20 ml of dichloromethane. Start stirring and the mixture was cooled to 0° C. Dissolve 2.30 g (10 mmol) of pentafluorobenzoyl chloride in 5 ml of dichloromethane and add this solution to the reaction mixture drop-wise. After addition, the mixture was allowed to warm to room temperature and stirred for another 5 h. Remove the solvent under vacuum and dissolve the residue in 100 ml of ethyl acetate. Wash the organic layer successively with brine, saturated aqueous sodium bicarbonate, brine, 0.1 N hydrochloric acid, and brine. After drying with magnesium sulfate, filtration, concentration under vacuum, and recrystallization (ethyl acetate/hexanes), 2.2 g of the title compound was obtained as a white solid in a yield of 78.6%. ¹H NMR (CDCl₃) δ 0.97 (d, J=6.79 Hz, 6H), 1.20 (d, J=6.74 Hz, 3H), 1.81 (m, J=6.72, 1H), 4.09 (m, 1H), 5.73 (br.s, 1H). MS (282.2, M+H).

### EXAMPLE 6

### N-(2-methylcyclohexyl)-2,3,4,5,6-pentafluoro-benzamide

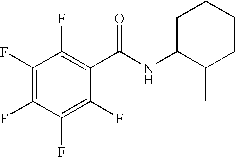

The title compound was prepared in a similar manner to Example 5 using 2-methylcyclohexyl amine and 2,3,4,5,6-pentafluorobenzoyl chloride. A white solid was obtained in a yield of 83%. ¹H NMR (CDCl₃) δ 0.94 (d, J=6.95 Hz, 30% of 3H), 1.01 (d, J=6.52 Hz, ˜70% of 3H), 1.09-1.23 (m, 2H), 1.28-1.44 (m, 2H), 1.54-1.66 (m, 1H plus ˜30% of 2H), 1.69-1.73 (m, ˜70% of 1H), 1.76-1.82 (m, 1H plus ˜70% of 1H), 1.90-1.98 (m, ˜30% of 1H), 2.05-2.09 (m, ˜70% of 1H), 3.63-3.70 (m, ˜70% of 1H), 4.24-4.29 (m, ˜30% of 1H), 5.87 (br.s, ˜70% of 1H), 5.96 (br.s, ˜30% of 1H). MS (308.1, M+H).

### EXAMPLE 7

### N-(2-methvlcvclohexvl)-2,3,4,5,6-pentafluoro-benzamide

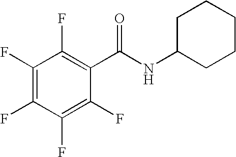

The title compound was prepared in a similar manner to Example 5 using cyclohexyl amine and 2,3,4,5,6-pentafluorobenzoyl chloride. A white solid was obtained in a yield of 63.8%. ¹H NMR (CDCl₃) δ 1.17-1.29 (m, 3H), 1.38-1.47 (m, 2H), 1.63-1.67 (m, 1H), 1.73-1.77 (m, 2H), 2.01-2.05 (m, 2H), 3.98 (m, 1H), 5.83 (br.s, 1H), MS (294.1, M+H).

### EXAMPLE 8

### N-(2, 6-dimethylcyclohexyl)-2,3,4,5,6-pentafluoro-benzamide

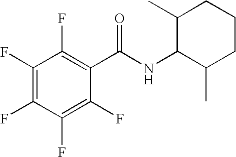

The title compound was prepared in a similar manner to Example 5 using 2, 6-dimethylcyclohexyl amine and 2,3,4,5,6-pentafluorobenzoyl chloride. A white solid was obtained in a yield of 95.3%. ¹H NMR (CDCl₃) δ 0.86-1.02 (m, 7H), 1.07-1.41 (m, 2H, plus 25% of 1H), 1.44-1.50 (m, 75% of 1H), 1.51-1.56 (m, 1H), 1.66-1.82 (m, 2H, plus ˜74% of 1H), 2.20-2.26 (m, ˜26% of 1H), 3.39-3.45 (m, ˜24% of 1H), 3.87-3.91 (m, ˜26% of 1H), 4.28-4.31 (m, ˜50% of 1H), 5.62-5.89 (3 br.s, 1H). MS (322.1, M+H).

### EXAMPLE 9

### N-(1,2-dimethylpropyl)-2,3,5,6-tetrafluoro-benzamide

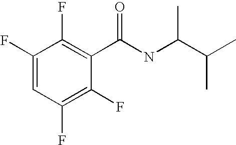

The title compound was prepared in a similar manner to Example 5 using 1,2-dimethylpropyl amine and 2,3,5,6-tetrafluorobenzoyl chloride. A white solid was obtained in a yield of 81.0%. ¹H NMR (CDCl₃) δ 0.97 (d, J=6.81 Hz, 6H), 1.20 (d, J=6.77 Hz, 3H), 1.80 (octet, J=6.72 Hz, 1H), 4.09 (m, 1H), 5.80 (br.s, 1H), 7.12 (m, 1H). MS (264.1, M+H).

### EXAMPLE 10

### N-(2-methylcyclohexyl)-2,3,5,6-tetrafluoro-benzamide

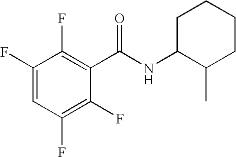

The title compound was prepared in a similar manner to Example 5 using 1-methyl cyclohexyl amine and 2,3,5,6-tetrafluorobenzoyl chloride. A white solid was obtained in a yield of 78.0%. ¹H NMR (CDCl₃) δ (0.96, d, J=6.96 Hz, ˜30% of 3H), 1.03 (d, J=6.54 Hz, ˜70% of 3H), 1.10-1.45 (m, 4H), 1.58-1.65 (m, 1H), 1,69-1.73 (m, 1H), 1.75-1.82 (m, 2H), 1.93-1.99 (m, ˜30% of 1H), 2.08-2.12 (m, ˜70% of 1H), 3.68-3.75 (m, ˜70% of 1H), 4.28-4.33 (m, ˜30% of 1H), 5.69 (br.s, ˜70% of 1H), 5.92 (br.s., ˜30% of 1H), 7.08-7.16 (m, 1H). MS (232.2, M+H).

### EXAMPLE 11

### Structure activity example of non-obviousness in structural correlation with sweet taste modality

During our invention process, we observed repeatedly that a small structural difference can cause dramatic change in the taste. For example, compound N-(2-methylcyclohexyl)-2,3,4,5,6-pentafluoro-benzamide is very sweet; moving the methyl group to the 4-position render the resulting N-(4-methylcyclohexyl)-2,3,4,5,6-pentafluoro-benzamide bitter; moving the methyl group to nitrogen render the resulting N-cyclohexyl-2,3,4,5,6-pentafluoro-N-methyl-benzamide tasteless. It has therefore been found that since addition, elimination or movement of even a single methyl group within a molecular skeleton can render a molecule sweet, bitter or tasteless, it is impossible to deem any structural modification to a known sweet molecule to obviously lead to another sweet molecule. In fact in most cases it does not.

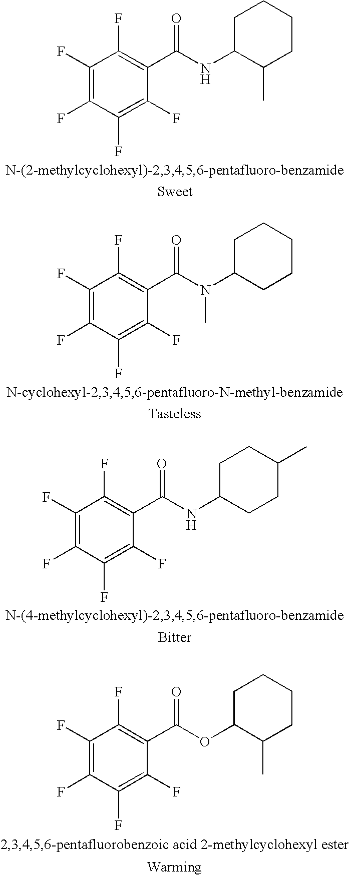

There is however an underlying theme, that being that an amide functionality is important. For example, 2,3,4,5,6-pentafluorobenzoic acid 2-methylcyclohexyl ester provides a burning sensation when tasted.

### EXAMPLE 12

### Comparisons of Taste Quality and Intensity

The following solutions in water were compared
8% sucrose
4% Sucrose
1 ppm N-(2-methylcyclohexyl)-2,3,4,5,6-pentafluoro-benzamide
5 ppm N-(2-methylcyclohexyl)-2,3,4,5,6-pentafluoro-benzamide
4% sucrose plus lppm N-(2-methylcyclohexyl)-2,3,4,5,6-pentafluoro-benzamide
4% Sucrose plus 5 ppm N-(2-methylcyclohexyl)-2,3,4,5,6-pentafluoro-benzamide
4% Sucrose has moderate sweetness and mouthfeel. Sweetness is rapid and fades quickly on rinsing of the mouth.

8% Sucrose has a good level sweetness and excellent mouthfeel. Sweetness is again rapid and fades quickly on rinsing of the mouth. At this concentration a slight bitterness is observed by some people.

1 ppm of N-(2-methylcyclohexyl)-2,3,4,5,6-pentafluoro-benzamide was considered to be equal in sweetness to about 1-2% sucrose, sweetness is not immediate but builds after a few seconds but is very clean, lacking bitterness, off-taste or negative attribute. The sweetness lacks any textural properties.

5 ppm of N-(2-methylcyclohexyl)-2,3,4,5,6-pentafluoro-benzamide was considered to be about equal in strength to about 4-6% sucrose. Again sweetness is not immediate but is very clean, lacking bitterness, off-taste or negative attribute. The sweet character lasts much longer than sucrose (up to 10 times longer) and remains clean in character throughout. The sweetness lacks the mouthfeel of sucrose solution of similar sweetness.

In combination 4% sucrose plus 1 ppm N-(2-methylcyclohexyl)-2,3,4,5,6-pentafluoro-benzamide was rated as sweet as 8% sucrose but it was noted that the maximum intensity sweetness was less immediate than noted with 8% sucrose. The sweetness was longer lasting and considered to be quite acceptable sweet character lacking bitterness. Additionally the textural properties of the sweet solution were considered acceptable.

In combination 4% sucrose plus 5 ppm N-(2-methylcyclohexyl)-2,3,4,5,6-pentafluoro-benzamide was rated as too sweet and too long lasting but the sweet profile while considered extreme was not bitter or obviously ‘unpleasant’.

The conclusion is that at low concentrations <4 ppm N-(2-methylcyclohexyl)-2,3,4,5,6-pentafluoro-benzamide has a synergistic effect with sucrose. The onset, duration and quality of the sweet taste profile are perceived as more than merely the sum of the individual taste experiences. An equivalent sweetness of solution lower in sugar can be made retaining the properties of sugar solution but lacking bitterness or lingering aftertaste.

TABLE I

| | PPM to | | |
|---|---|---|---|
| | Equate to 8% | | |
| Compound | sucrose | Profile | Other taste attributes |
| Sucrose | NA | Rapid sweet onset returns to | Bitter at high |
| | | baseline after 15 seconds | concentrations to |
| | | | some people |
| 53 | ~15 | Slow onset 2 × sucrose, | Clean |
| | | lingering sweetness | |
| Example 6 | −8 | Onset about 1.5 × sucrose, | Clean |
| | | lingers slightly longer than | |
| | | sucrose | |
| Example 10 | ~20 | More delayed onset and more | Slight bitterness |
| | | lingering | |
| 2,3,5,6-tetrafluoro- | ~12 | Onset about 1.5 × sucrose at this | Licorice, saccharin |
| 4-methyl-N-(2- | | concentration but sweetness is | like and soapy |
| methylcyclohexyl) | | long lasting >25 seconds, and | |
| benzamide | | unclean in character. | |
| Senomyx | | | |
| 40 | >100 | Poor sweet taste profile | Bitter at 20 ppm |
| 2,3,5,6-tetrafluoro- | 20 | Onset about 2.5 × sucrose, good | Clean |
| 4-methyl-N-(1,2- | | sweet character but very | |
| Dimethyl-propyl) | | lingering 30 seconds. | |
| benzamide | | | |
| Senomyx | | | |
| 2,3,5,6-tetrafluoro- | 50 | Onset about 2.0 × sucrose, weak | Very artificial tasting |
| 4-methyl-N-[(1S)- | | sweet character but very | with glycerizin notes |
| 1,2- | | lingering 40 seconds. | |
| Dimethylpropyl] | | | |
| benzamide | | | |
| Senomyx | | | |
| N-[(1R)-1,2,3,4- | ~25 | Onset about 1.5 × sucrose at this | Clean |
| tetrahydro-1- | | concentration but sweetness is | 1S isomer is tasteless |
| naphthalenyl]-3- | | long lasting >30 seconds | |
| furancarboxamide | | | |
| Senomyx | | | |
| 2,3,5,6-tetrafluoro- | 12 | Onset about 1.5 × sucrose, good | NHDC, licorice like |
| 4-methyl-N-(1,2- | | sweet character but very | off taste |
| Dimethyl-propyl) | | lingering >25 seconds | |
| benzamide | | | |
| Senomyx | | | |
| Example 2 | 10-12 | Slow onset 2.5 × sucrose. Clean | Clean |
| | | and short lasting sweetness <20 | |
| | | seconds | |

### EXAMPLE 13

### Comparison of N-(2-methylcyclohexyl)-2,3,4,5,6 pentafluoro-benzamide and 2,3,5,6-tetrafluoro-4-methyl-N-(2-methylcyclohexyl)benzamide

Two molecules were compared to each other based on sweetness. Both molecules were evaluated at 5 ppm in plain water. The first material, 2,3,5,6-tetrafluoro-4-methyl-N-(2-methylcyclohexyl)benzamide, was mild vegetative, green and very bitter with some sugary notes. The second molecule, N-(2-methylcyclohexyl)-2,3,4,5,6 pentafluoro-benzamide, was mouth coating, intense sweet, no bitter notes, and had a fast sweetness onset.

### EXAMPLE 14

### Comparison of N-(2-methylcyclohexyl)-2,3,4,5,6 pentafluoro-benzamide and 2,3,5,6-tetrafluoro-4-methyl-N-(2-methylcyclohexyl)benzamide in a beverage

An 8 brix, the measurement of the mass ratio of dissolved sugar in a liquid, beverage was prepared to evaluate two sweeteners, 2,3,5,6-tetrafluoro-4-methyl-N-(2-methylcyclohexyl)benzamide-(sweetener A) and N-(2-methylcyclohexyl)-2,3,4,5,6 pentafluoro-benzamide)—(sweetener B). The objective was to determine if the sweeteners enhanced an 8 brix beverage to make it taste more similar to a 10 brix beverage. There were four samples evaluated an 8 brix beverage blank, sweetener A in 8 brix beverage, sweetener B in 8 brix beverage and a 10 brix beverage blank. Both the sweeteners were evaluated at 4 ppm. The beverage samples were evaluated by a trained technical panel. The panel evaluated the sweetness profile and the sweet intensity. The intensity was rated on a ten point scale with nine being the most sweetness and zero having no sweetness. The over all comments were that sweetener A was sour and slightly sweeter than the 8 brix blank. The sweetener B, was sweeter, more mouthwatering, had a slight astringent aftertaste and had a similar sweetness profile to the 10 brix beverage. The sweet intensity ratings were as follows (see Table 2) sample A rated 6, sample B rated 7, sample C rated 9 and sample D rated 9. In conclusion, the sweetener B made an 8 brix beverage taste more like a 10 brix beverage.

TABLE 2

| | |
|---|---|
| Sample A | 8 Brix beverage |
| Sample B | 8 Brix beverage plus sweetener A |
| Sample C | 8 Brix beverage plus sweetener B |
| Sample D | 10 Brix beverage |

### EXAMPLE 14

### Comparison of N-(2-methylcyclohexyl)-2,3,4,5,6 pentafluoro-benzamide and 2,3,5,6-tetrafluoro-4-methyl-N-(2-methylcyclohexyl)benzamide in a muffins

The sweeteners 2,3,5,6-tetrafluoro-4-methyl-N-(2-methylcyclohexyl)benzamide (sweetener A) and N-(2-methylcyclohexyl)-2,3,4,5,6 pentafluoro-benzamide -(sweetener B) were both evaluated in muffins. They were evaluated along with a blank muffin sample. Both sweeteners were evaluated at 10 ppm in the muffin. The samples were evaluated by a trained technical panel. They were asked to evaluate the profile and to give a rating between 0-9 on the sweetness intensity. The rating scale ranged from zero for no sweetness to 9 for the most sweetness. The sweetener A sample was described as slightly sweet, not much difference in profile to the blank muffin sample. The sweetener B sample was described as sweeter, long lasting sweetness and mouthwatering. The samples were rating on sweet intensity (see Table 3). The samples ratings were as follows; sample A rated 5, sample B rated 6, sample C rated 8. In conclusion the sample with sweetener B had more sweet intensity than the sample containing sweetener A.

TABLE 3

| | |
|---|---|
| Sample A | Control |
| Sample B | Sweetener A |
| Sample C | Sweetener B |

### EXAMPLE 15

### Comparison of N-(2-methylcyclohexyl)-2,3,4,5,6 pentafluoro-benzamide and 2,3,5,6-tetrafluoro-4-methyl-N-(2-methylcyclohexyl)benzamide in a beverage in combination with Aspartame

The sweeteners Benzamide, 2,3,5,6-tetrahydrofluoro-4-methyl-N-(2-methylcyclohexyl)—(sweetener A) and Benzamide, 2,3,4,5,6-pentafluoro-N-(2-methylcyclohexyl)—(sweetener B) were both evaluated in a cola beverage that contained Aspartame. The purpose of this experiment was to evaluate the sweeteners profile in a sweetener blend. There were three separate samples evaluated, see Table 3. The samples were evaluated by a trained technical panel. The sweetness profile was rated on a scale from 0-9, with 0 being the worst and 9 the best. The first sample, Sample A, containing only aspartame at 300 ppm was rated a 4. The comments were that the sweetness in the beverage spiked and then disappeared. The second sample, Sample B, contained Aspartame at 300 ppm and sweetener A at 3 ppm. The comments were that the sweetness was well rounded, lingered, and gave mouthfeel. The rating for sample B was a 9 for sweetness. The third sample, sample C, contained Aspartame at 300 ppm and sweetener B at 3 ppm. The comments were that the sweetness had more impact than Sample A, but not as intense as Sample B. The rating for sample C was a 6 on sweetness.

TABLE 4
Cola Beverage samples

| | |
|---|---|
| Sample A | Aspartame 300 ppm |
| Sample B | Aspartame 300 ppm + Sweetener A |
| Sample C | Aspartame 300 ppm + Sweetener B |

### EXAMPLE 16

### Sweetener Blends

A comparison of the synergistic effects the Benzamide, 2,3,4,5,6-Pentafluoro-N-(2-methylcyclohexyl)—(sweetener A) in combination with acesulfame-k, herein referred to as Ace-K and Sucralose was compared with the effects of Benzamide, 2,3,5,6-tetrafluoro-4-methyl-N-(2-methylcyclohexyl)—(sweetener B) in combination with the same commercial sweeteners. The samples were evaluated by a trained technical panel. The first sample was Ace-K at 100 ppm. The comments were delayed sweetness, some off notes, astringent and bitter. The comments for the combination of sweetener A at 3 ppm with Ace-K at 100 ppm was a strong sweetness upfront, bitter, enhanced bitterness in combination. The comments for the blend of the sweetener B at 3 ppm in combination with Ace-K at 100 ppm was delayed sweetness, astringency, not as strong a sweetness as the combination with sweetener A. The overall group rating on a scale from 1-9 with 1 being the worst and 9 the best was a 9 for the blend with sweetener A and 7 for the blend with sweetener B.

A similar evaluation with done with Sucralose as the commercial sweetener. An evaluation of Sucralose at 60 ppm was done in plain water. The comments were sweet onset, mouthwatering, sweetness lingers. A comparison of the synergistic effects of sweetener A at 3 ppm in combination with Sucralose at 60 ppm was evaluated against sweetener B in the same combination. The results of the evaluation of the blend with sweetener A was enhanced sweetness, more rounded, no bitter, very strong sweet profile. The results of the evaluation of the blend with sweetener B was not as sweet as the combination with sweetener A, delayed sweetness. The blends were evaluated on a scale for 1 to 9. The blend using sweetener A was given a rating of 9 and the blend using sweetener B was given a rating of 7.

### EXAMPLE 17

### Sweetener Blends

Sweetener blend 1 contains Benzamide,2,3,4,5,6-pentafluoro-N-(2-methylcyclohexyl)—at 2 ppm and Benzamide, 2,3,5,6-tetrafluro-4-methyl-N-(2-methylcyclohexyl)—at 2 ppm. The taste comments for the blend was sweetness upfront, drying, some mouthwatering, lingers artificial sweetener.

Sweetener Blend 2 contains Benzamide,2,3,4,5,6-pentafluoro-N-(2-methylcyclohexyl)—at 2 ppm, Benzamide, 2,3,5,6-tetrafluro-4-methyl-N-(2-methylcyclohexyl)—at 2 ppm and Sucralose at 60 ppm. The taste comments were sweet upfront, natural sugar taste, mouthwatering, lingers sweet, well rounded profile.

Sweetener Blend 3 contains Benzamide,2,3,4,5,6-pentafluoro-N-(2-methylcyclohexyl)—at 2 ppm, Benzamide, 2,3,5,6-tetrafluro-4-methyl-N-(2-methylcyclohexyl)—at 2 ppm and Steavia at 1000 ppm. The taste comments were sweet upfront, slight bitter, anise note, lingers sweet, drying.

What is claimed is:

1. A compound benzamide,2,3,4,5,6-pentafluoro-N-(2-methylcyclohexyl)-.

2. An ingestible product or oral composition containing the compound of claim 1 in an effective amount.

3. A sweetening composition comprising the compound of claim 1 and a carrier.

4. A method of sweetening a substance comprising the steps of incorporating the compound of claim 1 in an effective amount into the substance.

5. A sweetener composition comprising the compound of claim 1 and an artificial sweetener.

6. The composition of claim 5 wherein the artificial sweetener is selected from the group consisting of aspartame, saccharin, acesulfame-k, cyclamate, sucralose, and alitame, and a mixture thereof.

7. The sweetener composition of claim 6 wherein the compound of claim 1 is present from about 0.001 ppm to about 100 ppm.

8. The sweetener composition of claim 6 wherein the compound of claim 1 is present from about 0.01 ppm to about 30 ppm.

9. The sweetener composition of claim 6 wherein the compound of claim 1 is present from about 0.05 ppm to about 15 ppm.

10. The sweetener composition of claim 6 wherein the compound of claim 1 is present from about 0. 1 ppm to about 5 ppm.

11. A sweetener composition comprising the compound of claim 1 and natural sweetening compositions selected from the group consisting of sucrose, glucose, fructose, lactose, maltose, erythritol, isomalt, lactitol, mannitol, sorbitol, xylitol, flavanoid, protein sweetener and mixtures thereof.

12. The sweetener composition of claim 11 wherein the compound of claim 1 is present from about 0.001 ppm to about 100 ppm.

13. A sweetener composition comprising the compound of claim 1 and benzamide, 2,3,5,6-tetrafluro-4-methyl-N-(2-methylcyclohexyl)-.

14. A sweetening composition comprising the compound of claim 1, benzamide, 2,3,5,6-tetrafluro-4-methyl-N-(2-methylcyclohexyl)—and Sucralose.

15. A sweetening composition comprising the compound of claim 1, Benzamide, 2,3,5,6-tetrafluro-4-methyl-N-(2-methylcyclohexyl)—and Steavia.

16. A method for modulating the taste of a consumable product comprising providing at least one consumable product, or a precursor thereof, and combining the consumable product or precursor thereof with at least a sweet flavor-modulating amount of the compound benzamide,2,3,4,5,6-pentafluoro-N-(2-methylcyclohexyl)-.

17. The method of claim 16 wherein the modified consumable product is a food for animal consumption

18. The method of claim 16 wherein the modified consumable product is a food for human consumption.

19. The method of claim 16 wherein the modified consumable products is selected from the group consisting of confectioneries, bakery products, ice cream, dairy products, sweet and savory snack bars, meal replacement products, ready meals, soups, pastas, noodles, canned foods, frozen foods, dried foods, chilled foods, oils and fats, baby foods, and spreads.

20. The method of claim 16 wherein the modified consumable product comprises one or more meats, poultry, fish, vegetables, grains and fruits.

21. The method of claim 16 wherein the modified consumable product is selected from the group consisting of a frozen food, an uncooked food, a fully cooked food and a partially cooked food.

22. The method of claim 16 wherein the modified consumable product is a snack food.

23. The method of claim 16 wherein the modified consumable product is selected from the group consisting of a cooking aid product, a meal solution product, a meal enhancement product, a seasoning, and a seasoning blend.

24. The method of claim 16 wherein the modified consumable product is selected from the group consisting of a cake, cookie, pie, candy, chewing gum, gelatin, ice cream, sorbet, pudding, jam, jelly, salad dressing, condiment, cereal, canned fruit, and fruit sauces.

25. The method of claim 16 wherein the modified consumable product is selected from the group consisting of a beverage, a beverage mix, and a beverage concentrate.

26. The method of claim 16 wherein the modified consumable product is selected from the group consisting soda and juice.

27. The method of claim 16 wherein the modified consumable product is an alcoholic beverage.

28. The method of claim 16 wherein the modified consumable product is an oral hygiene product.

29. The method of claim 16 wherein the compound is present in the modified consumable product in a concentration from about 0.001 ppm to about 100 ppm.

30. The method of claim 16 wherein the compound is present in the modified consumable product in a concentration from about 0.01 ppm to about 30 ppm.

31. The method of claim 16 wherein the compound is present in the modified consumable product in a concentration from about 0.05 ppm to about 15 ppm.

32. The method of claim 16 wherein the compound is present in the modified consumable product in a concentration from about 0.1 ppm to about 5 ppm.

33. The method of claim 16 wherein the compound is present in the modified consumable product in a concentration from about 0.05 ppm to about 15 ppm.

34. The method of claim 16 further comprising benzamide, 2,3,5,6-tetrafluro-4-methyl-N-(2-methylcyclohexyl)-.

35. The method of claim 16 further comprising benzamide, 2,3,5,6-tetrafluro-4-methyl-N-(2-methylcyclohexyl)—and Sucralose.

36. The method of claim 16 further comprising benzamide, 2,3,5,6-tetrafluro-4-methyl-N-(2-methylcyclohexyl)—and Steavia.
